(19) United States
(12) Patent Application Publication
(10) Pub. No.: US 2017/0342365 A1
(43) Pub. Date: Nov. 30, 2017

(54) CLOSED-SYSTEM CULTURE VESSEL, TRANSPORT METHOD, AND AUTOMATED CULTURING DEVICE

(71) Applicant: Hitachi, Ltd., Tokyo (JP)

(72) Inventors: Takayuki NOZAKI, Tokyo (JP); Guangbin ZHOU, Tokyo (JP); Masaharu KIYAMA, Tokyo (JP); Masakazu SUGAYA, Tokyo (JP)

(21) Appl. No.: 15/536,673

(22) PCT Filed: Mar. 27, 2015
(86) PCT No.: PCT/JP2015/059716

§ 371 Date: Jun. 16, 2017

(51) Int. Cl. C12M 1/00; C12M 1/36; C12M 1/34; C12M 1/12; C12M 3/00

(52) U.S. Cl. CPC C12M 23/58; C12M 23/02; C12M 23/34; C12M 41/36; C12M 41/14; C12M 1/125; C12M 45/20; C12M 41/48; C12M 23/38; C12M 23/48; C12M 29/00

(57) ABSTRACT

To provide a closed system culture vessel for realizing culturing in which an aseptic state is maintained at the time of culturing, and making the reduction of the shear stress generated during conveyance after manufacturing possible. A closed system culture vessel 201 holds a second vessel 302 consisting of an insert vessel on the inside of a first vessel 301, and a lid portion 303 of the closed system culture vessel 201 forms a closed space with the first vessel 301 and has a convex portion 305 contacting the entire outer circumference of the second vessel 302. During conveyance, the closed system culture vessel is conveyed in a state in which the culture medium for conveyance is filled within the second vessel 302.

### TECHNICAL FIELD

The present invention relates to a closed system culture vessel for culturing cells or tissues by an automated culture apparatus by an automatic operation, and conveying after culturing.

### BACKGROUND ART

The regenerative medical treatment in which the functions of organs and the like are recovered using the regenerative tissue manufactured using cells as the raw material is expected to become a fundamental medical treatment method for disease for which there have been no medical treatment methods. The object for treatment includes many different types such as the skin, cornea, esophagus, heart, bone, cartilage and the like, and examples of clinical use have also been increasing rapidly. The manufacturing process of the regenerative tissues performs processes such as separating, purifying, amplifying and organizing a biological sample acquired from the patient himself/herself or another person in a Cell Processing Center (CPC). Therefore, in order to manage the CPC, huge costs and human resources with specialized culture techniques are required. Additionally, since all manufacturing processes are conducted manually, there is a limit to the increase of the production amount of the regenerative tissues. The low productivity and the high production costs hinder the spreading of the regenerative medical treatment, thus, the automation of the culturing operations among the manufacturing processes, specifically, the operations which require labor and cost has been sought. By conducting the automation of the culturing operations, a reduction in the workforce and the costs are achieved, and mass production becomes possible.

An example of the automated culture apparatus is an apparatus which automatically handles a closed-system culture vessel having a closed space. The closed-system culture vessel is in a state which is consistently connected with passage tubes and the like at the time of culturing, and the automated culture apparatus automatically executes cell seeding, culture medium exchange, microscopic observation and the like in a state in which the closed property of the culture space is maintained. The advantage that the risk of biological contamination is reduced is obtained thereby. After production, the regenerative tissues are conveyed to an operating room where the transplantation is performed. At this time, it is necessary to convey the regenerative tissues in a state in which the quality of the regenerative tissues is maintained.

One example of the closed system culture vessel which uses an automated culture apparatus is disclosed in Patent Literature 1. Herein, the closed-system culture vessel has a two-layer culture vessel, and the passages for supplying or discharging the culture medium and the like are constantly coupled therewith. Further, in Patent Literature 2, the regenerative tissues are made so as not to move within a packing vessel during conveyance by storing the regenerative tissues after production within a carrier, and fixing the carrier the inside of packing vessel, and as a result, the prevention of damage to the regenerative tissues during conveyance is possible.

### CITATION LIST

### Patent Literature

Patent Literature 1: WO12/008368
Patent Literature 2: Japanese Unexamined Patent Application Publication No. 2007-119033

### SUMMARY OF INVENTION

### Technical Problem

As shown in Patent Literature 1, the production of the regenerative tissues which uses the closed-system culture vessel within an automated culture apparatus has already been achieved. It is also possible to manufacture various types of regenerative tissues by selecting the number of layers of the culture vessel in accordance with the cell type. Further, this technique uses a closed system culture vessel, thus, the maintenance of an aseptic state at the time of culturing is possible. Meanwhile, as shown in Patent Literature 2, storing the produced regenerative tissues in a vessel for conveyance, and conveying in a state in which the regenerative tissues are filled in a culture medium for conveyance has already been achieved. The quality of the regenerative tissues may be filled in a state maintained in the culture medium for conveyance and by maintaining the temperature during conveyance at an optimal value.

However, there is the risk that in conducting an operation for transferring the regenerative tissues to a different vessel from during the production thereof after production by the automated culture apparatus, the cleanliness that the regenerative tissues had during production is lost. In addition, there is the risk that during conveyance, the vessel for conveyance which stores the regenerative tissues may receive oscillations and impacts, and the culture medium for conveyance will be agitated on the inside and a shear stress will be produced in the regenerative tissues, and the quality of the regenerative tissues after conveyance will decrease.

Meanwhile, when the culture medium for conveyance is filled and conveyed only as a liquid phase, the decrease of the shear stress is possible, but when the regenerative tissues are taken out from the vessel for conveyance after conveyance, the culture medium for conveyance may leak outside the vessel for conveyance. The production is conducted the inside of the CPC, and thus, is handled in a clean environment, but during conveyance, the vessel passes through environments in which the cleanliness is not managed. Therefore, the outside of the vessel for conveyance after conveyance is unclean. As mentioned above, if the culture medium for conveyance leaks when taking out the regenerative tissues, there is the risk that the regenerative tissues will be biologically contaminated by contact with the outside of the unclean vessel for conveyance via the culture medium for conveyance.

The object of the present invention is to provide a closed system culture vessel which makes it possible to solve the above problem, maintain the cleanliness, and control the shear stress generated during conveyance, a conveyance method thereof, and an automated culture apparatus.

### Solution to Problem

To attain the aforementioned object, the present invention constitutes a closed system culture vessel for holding cells including a first vessel, a second vessel arranged within the first vessel and having a porous membrane coupling an opening with the first vessel side, and a lid portion for sealing the first vessel, wherein the lid portion forms a closed space with the first vessel, and the lid portion is in contact with the opening of the second vessel over the entire circumference, thus, controlling transfer of a liquid held within the second vessel into the first vessel to make it possible to hold the liquid in the second vessel.

Further, to attain the aforementioned object, the present invention constitutes a closed system culture vessel for holding cells including a first semi-open space, a second semi-open space arranged in order to surround the first semi-open space, and a lid portion for sealing the first semi-open space, wherein the lid portion forms a closed space with the first semi-open space, the lid portion is in contact with the opening of the second semi-open space over the entire circumference, transferring the liquid held within the second semi-open space into the first semi-open space surrounding the second semi-open space is controlled by the contact between the lid portion and the opening of the second semi-open space, and makes it possible to hold the liquid in the second semi-open space.

Furthermore, to attain the aforementioned object, the present invention constitutes a conveyance method of the closed system culture vessel for holding cells, wherein the closed system culture vessel is provided with a first vessel, a second vessel arranged within the first vessel and having a porous membrane coupling the opening with the first vessel side, and the lid portion for sealing the first vessel, wherein the lid portion has a connector part for performing the feeding of the liquid or gas to the first vessel and the second vessel, and a convex portion connecting the second vessel with the opening, the convex portion is provided with fine holes between the first vessel and the second vessel, the lid portion forms a closed space with the first vessel, the lid portion is in contact with the entire circumference of the opening of the second vessel, controls the transferring of the liquid being held within the second vessel within the first vessel to make it possible to hold the liquid in the second vessel, and supplies the culture medium for conveyance into the second vessel to the height of the fine holes, and performing the conveyance after the passage tubes coupled to the connector part are closed.

Furthermore, to attain the aforementioned object, the present invention is an automated culture apparatus for culturing cells including a holding part for holding a liquid or a gas required for culturing, a closed system culture vessel for holding the liquid or gas supplied from the holding part, a passage part provided with a plurality of passages connecting the holding part with the closed system culture vessel, a control unit for controlling the passage part, and for controlling so as to supply or discharge the liquid or gas from the holding part to the closed system culture vessel, wherein the closed system culture vessel is provided with a first vessel, a second vessel arranged within the first vessel, and having a porous membrane coupling the opening with the first vessel side, and a lid portion for sealing the first vessel, the lid portion has a connector part for performing the supply or discharge of the liquids or gases to the first vessel and the second vessel and the lid portion forms a closed space with the first vessel, and the lid portion is in contact with the entire circumference of the opening of the second vessel, controlling transfer of the liquid supplied to and held within the second vessel to the first vessel to make it possible to hold the liquid in the second vessel.

### Advantageous Effects of Invention

The present invention can provide a closed system culture vessel for maintaining the cleanliness, and making the control of the shear stress generate during conveyance possible, a conveyance method thereof, and an automated culture apparatus.

### BRIEF DESCRIPTION OF DRAWINGS

FIG. 1 is a drawing illustrating an exemplary configuration of the automated culture apparatus according to a first embodiment.

FIG. 2 is a drawing illustrating an example of the path circuit including the closed system culture vessel according to the first embodiment.

FIG. 3A is a drawing illustrating an exemplary configuration of the closed system culture vessel according to the first embodiment.

FIG. 3B is a drawing illustrating an exemplary configuration of the closed system culture vessel according to the first embodiment.

FIG. 4A is a drawing illustrating the closed system culture vessel at the time of culturing, during conveyance, and when the regenerative tissues are taken out according to the first embodiment.

FIG. 4B is a drawing illustrating the closed system culture vessel at the time of culturing, during conveyance, and when the regenerative tissues are taken out according to the first embodiment.

FIG. 4C is a drawing illustrating the closed system culture vessel at the time of culturing, during conveyance, and when the regenerative tissues are taken out according to the first embodiment.

FIG. 4D is a drawing illustrating the closed system culture vessel at the time of culturing, during conveyance, and when the regenerative tissues are taken out according to the first embodiment.

FIG. 4E is a drawing illustrating the closed system culture vessel at the time of culturing, during conveyance, and when the regenerative tissues are taken out according to the first embodiment.

FIG. 4F is a drawing illustrating the closed system culture vessel at the time of culturing, during conveyance, and when the regenerative tissues are taken out according to the first embodiment.

FIG. 5 is a drawing illustrating an example of the control mechanism of the automated culture apparatus according to the first embodiment.

FIG. 6 is a drawing illustrating the process flow during the operation of the automated culture apparatus according to the first embodiment.

FIG. 7A is a drawing illustrating an exemplary configuration of the closed system culture vessel according to a second embodiment.

FIG. 7B is a drawing illustrating another exemplary configuration of the closed system culture vessel according to the second embodiment.

FIG. 7C is a drawing illustrating another exemplary configuration of the closed system culture vessel according to the second embodiment.

FIG. 7D is a drawing illustrating another exemplary configuration of the closed system culture vessel according to the second embodiment.

FIG. 8A is a drawing illustrating an exemplary configuration for transferring and conveying the insert vessel after culturing by a manual operation according to a third embodiment.

FIG. 8B is a drawing illustrating another exemplary configuration for transferring and conveying the insert vessel after culturing by a manual operation according to the third embodiment.

FIG. 9 is a drawing illustrating an exemplary configuration for conveying without transferring the insert vessel after culturing by manual operation according to a fourth embodiment.

FIG. 10A is a drawing illustrating an exemplary configuration of the closed system culture vessel using the cover member according to a sixth embodiment.

FIG. 10B is a drawing illustrating another exemplary configuration of the closed system culture vessel using the cover member according to the sixth embodiment.

FIG. 11 is a drawing illustrating an exemplary configuration of the closed system culture vessel using the culture dish according to a seventh embodiment.

### DESCRIPTION OF EMBODIMENTS

The various embodiments of the present invention will be explained below accompanying the drawings.

### First Embodiment

A first embodiment is an example of a closed system culture vessel which can maintain an aseptic state at the time of culturing, and which can reduce shear stress and maintain the cleanliness during conveyance, a conveyance method, and an automated culture apparatus. The closed system culture vessel of the present embodiment is provided with a first vessel, a second vessel arranged within the first vessel and having a porous membrane coupling the opening with the first vessel side, and a lid portion for sealing the first vessel, wherein the lid portion forms a closed space with the first vessel and the lid portion is in contact with the entire circumference of the opening of the second vessel, and the lid portion has a connector part for performing the feeding of the liquid or gas necessary for culturing to the first vessel and the second vessel, and a convex portion connecting the second vessel with the opening, and the convex portion is further provided with the fine holes between the first vessel and the second vessel, thus, controlling the liquid held within the second vessel from being transferred into the first vessel makes it possible to hold the liquid in the second vessel.

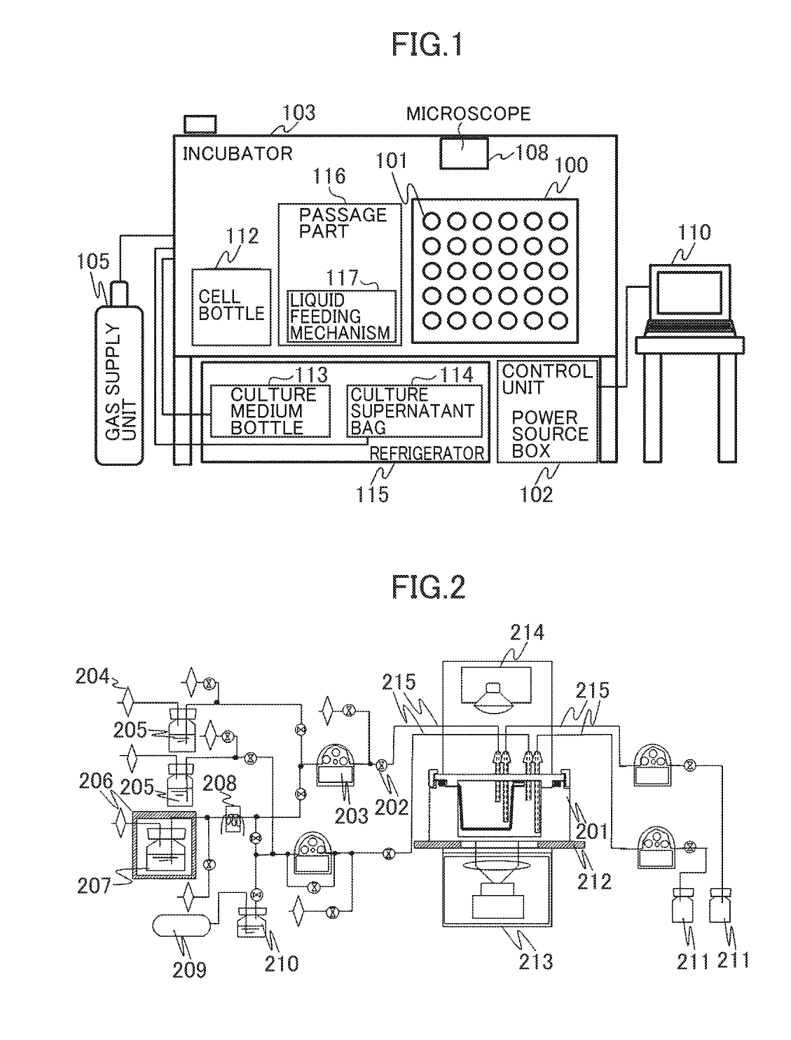

First, the constitutional elements of the automated culture apparatus for automatically performing culturing using the closed-system culture vessel of the present embodiment will be explained using FIG. 1. The automated culture apparatus includes an incubator 103 which is the space for culturing the cells at 37° C. which is the culturing temperature, a culture vessel unit 100 arranged in the incubator 103, a microscope 108, a cell bottle 112, a passage part 116 having a liquid feeding mechanism 117 for feeding cells and the culture medium, a refrigerator 115 for maintaining a culture medium bottle 113 wherein the culture medium is placed on the inside and a culture supernatant bag 114 for recovering the culture supernatant, a gas supply unit 105 for supplying air containing 5% C2 to the closed system culture vessel 101 within the culture vessel unit 100, and a power source box, and includes a control unit 102, a control terminal 110 and the like for controlling the automated culture apparatus.

The number of closed system culture vessels 101 within the culture vessel unit 100 may be one or more. Further, the closed system culture vessel 101 is constantly coupled with the cell bottle 112, the culture medium bottle 113, the culture supernatant bag 114, and the like via the passage tubes, etc., within the passage part 116. Note that, in the description, there are cases where the cell bottle 112, the culture medium bottle 113, and furthermore, the culture supernatant bag 114 are collectively referred to as the holding part. The cells within the closed system culture vessel 101 are suitably observed by the microscope 108. The liquid feeding mechanism 117 of the passage part 116 is constituted by a drive system such as a solenoid valve or a tube pump for feeding the culture medium, etc., to the closed system culture vessel 101.

The automated culture apparatus performs cell seeding by feeding the cell suspension to the closed system culture vessel 101, culturing which maintains the temperature and the gas environment, a culture medium exchange for discharging the old culture medium and supplying a new culture medium, observation of the cells by a microscope, and the like. The processes performed by the automated culture apparatus are set as cell seeding, culture medium exchange, culturing, and microscopic observation in the present example, but it is obvious that the invention is suitable even if a part of the process is changed by a manual operation.

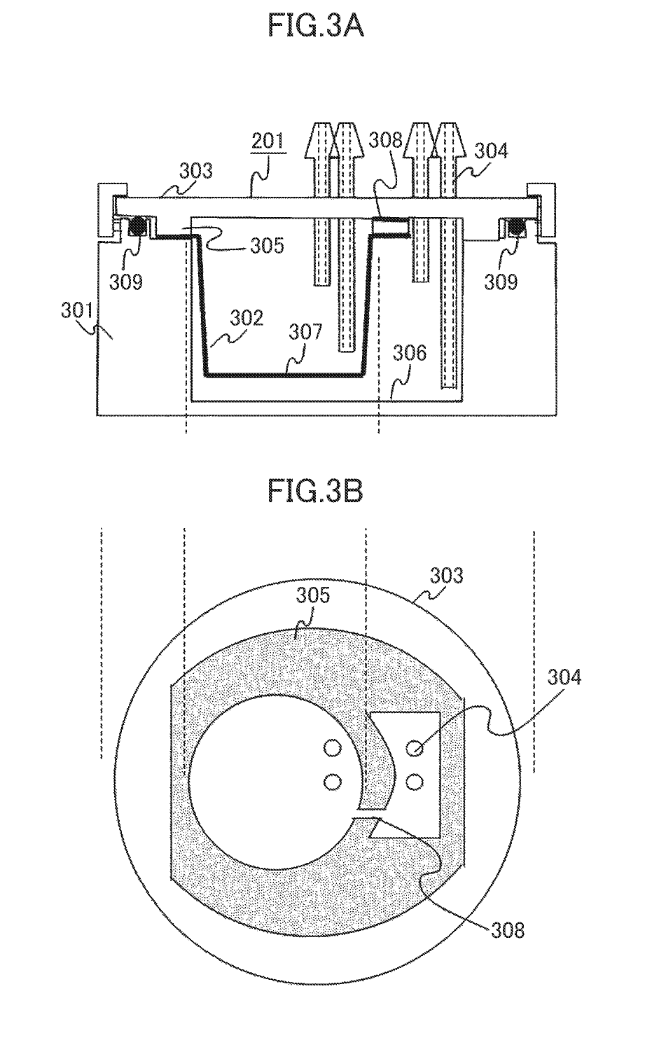

FIG. 2 shows an example of the path circuit when using the closed system culture vessel of the present embodiment to produce regenerative tissues by the automated culture apparatus. The path circuit is provided with liquid feeding control means for controlling the feeding relating to the supply or discharge of the culture medium to the closed system culture vessel 201 to be described later and which corresponds to the closed system culture vessel 101 of FIG. 1. Gas feeding control means is also provided when directly feeding within the closed system culture vessel 201. In the case of a configuration such as when a part of the closed system culture vessel 201 is made as a gas permeation film, the gas is supplied via the gas permeation film. In this case, however, the incubator itself is the gas feeding means. An example of the flow path circuit of FIG. 3 is shown in the former configuration. Further, in the example, two cell bottles 205 corresponding to the cell bottle 112 of FIG. 1 are used, but this example is the culturing of epithelial cells by a feeder layer method. Further, in the drawing, one closed system culture vessel 201 is shown, but as shown in FIG. 1, the automatic culturing by a plurality of the closed system culture vessels simultaneously is possible by disposing the closed system culture vessels in parallel.

As shown in FIG. 2, the closed system culture vessel 201 is a two-layer structure, and supplying and discharging are performed in each layer, thus, four passage tubes 215 are connected to one closed system culture vessel. The opening and closing of the passage tubes 215 is controlled by the operation of the solenoid valve 202 operating from the outside of the passage tubes. Further, the driving of the liquid and the gas into the passage tubes is controlled by the operation of the pump 203. The pump to be used is, for example, a roller pump. Further, a filter 204 is arranged in the path circuit, and acquires a gas from outside the passages and controls the atmospheric pressure inside the passage. The filter 204 of a quality which, for example, does not allow passage of particle size of 0.22 μm or more is used.

In the present embodiment, two cell bottles 205 are used, and are held in a state in which, for example, epithelial cells which are the object for culturing are suspended in a culture medium in one bottle, and, for example, feeder cells are suspended in a culture medium in the other bottle. A culture medium bottle 207 holding the culture medium similarly corresponding to the culture medium bottle 113 is arranged within the refrigerator 206 corresponding to the refrigerator 115 of FIG. 1, and the cells at the time of culturing are fed to the closed system culture vessel 201 after the culture medium is preheated by a preheating mechanism 208.

The gas supply unit 209 is, for example, a gas cylinder filled with carbon dioxide gas pressurized at optimum concentration. A humidifying bottle 210 for humidifying the gases fed from the gas supply unit 209 to an optimum temperature is arranged downstream of the gas supply unit 209. The purpose of the gas supply unit 209 and the humidifying bottle 210 is the implementation of periodic gas exchange for preventing the pH value of the culture medium from changing during culturing, and in this case, the prevention of the concentration of the culture medium component due to the evaporation of moisture from the culture medium.

The culture supernatant bag 211 for recovery of the culture supernatant after the culture medium exchange is arranged downstream of the closed system culture vessel 201. This culture supernatant bag 211 can be arranged within the refrigerator 206 in the same manner as the culture supernatant bag 114 of FIG. 1. Further, while not shown in the drawing, the culture supernatant recovery bag is arranged in a position parallel to the culture supernatant bag 211, the culture supernatant is aseptically collected in the middle of culturing, and the normality of the culture state can be verified by culture medium component analysis.

The microscope observation unit 213 is arranged below the stage 212 on which the closed system culture vessel 201 is arranged. A light irradiation portion 214 which is a part of the microscope observation unit 213 is arranged above the closed system culture vessel. The microscope observation unit 213 and the light irradiation portion 214 correspond to the microscope 108 of FIG. 1. Further, the stage 212 can adjust the observation location within the closed-system culture vessel 201 by an up/down drive device of the microscope observation unit 213.

Using the aforementioned configuration, cell seeding is performed by the feeding of the cell suspension from the cell bottle to each layer of the closed system culture vessel. Then, the culture medium exchange and the gas exchange are performed at an appropriate frequency according to the cell type. In the middle of culturing, microscopic observation is performed according to a predetermined schedule, and the growth conditions of the cells are verified. According to need, the culture supernatant recovered after the culture medium exchange is used and evaluations such as of the metabolism are performed. The regenerative tissues are finally obtained through the aforementioned processes.

The fundamental constitutional elements of the closed-system culture vessel of the present embodiment will be explained using FIG. 3. The closed system culture vessel holds inside thereof the cells and the regenerative tissues which are the biological sample. Therefore, sterilization is performed by a sterilization process before culturing. When the material is, for example, polystyrene, the material may be sterilized by subjecting the material to a sterilization operation by a γ-ray radiation process, an ethylene oxide gas process, or the like prior to use. The aforementioned example uses polystyrene, but it goes without saying that any compound is suitable as along as the sterilization of the material which is not harmful to the biological sample is possible. Further, it is preferable that the material is of a quality for a medical application which does not produce harmful substances.

The closed system culture vessel 201 is a two-layer structure as shown in the schematic illustration of FIG. 3A, and mainly consists of a first vessel 301, a second vessel 302, and a lid portion 303. A packing unit 309 for sealing by the lid portion 303 is formed in the periphery of the top of the first vessel 301. The second vessel 302 is a vessel having an opening, and for example, is an insert vessel which has been generally used in cell culturing by manual culturing. The closed system culture vessel 201 forms a closed space with the exception of the connector part 304 which can be connected with the passage tubes 215. The two-layer culturing of, for example, epithelial cells which uses feeder cells is made possible thereby. At the time of automatic culturing, the closed system culture vessel 201 is normally connected to the path circuit described in FIG. 2 via the passage tubes 215. The passage tubes 215 are mounted to the connector part 304. As previously stated, because this is two-layer culturing, the connector part for the supply and the connector part for the discharge are arranged in one layer, and thus, there are a total of four connector parts 304.

FIG. 3B shows the back side of the lid portion 303. FIG. 3B shows the convex portion 305 of the lid portion 303 and four connector parts 304. Apart of the convex portion 305 may maintain the culture surface 306 of the first vessel 301 in parallel with the culture surface 307 of the second vessel 302 by sandwiching the first vessel 301 with a part of the second vessel 302. Incidentally, in the configuration of the present embodiment, the culture surface 307 is a porous membrane having a hole coupling the first vessel side with the second vessel side. For example, a growth factor discharged by the feeder cells which are cultured at the culture surface 306 passes through the hole of the culture surface 307 which is the porous membrane, reaches the epithelial cells which are cultured on the culture surface 307, and the epithelial cells undergo growth such as proliferation thereby.

Further, the convex portion 305 has fine holes 308 which make the movement of gas between the first vessel 301 and the second vessel 302 possible in a state which the closed system culture vessel 201 is assembled. The fine holes 308 formed by the convex portion 305 may be of a size in the range at which pressure loss does not occur in the gas movement under a normal pressure such as a hole having a width of 1 μm and a depth of 1 μm is formed in the convex portion 305 may be provided as an example. The radius can prevent pressure loss when in the range from approximately 1 μm to several 10 μm.

Furthermore, a part of the convex portion 305 is in contact with the entirety of the outer circumference of the opening of the second vessel 302. A space which does not allow the movement of gases with the exception of the hole of the culture surface 307 which is a porous membrane and the fine holes 308 having the convex portion 305 is formed by the second vessel 302 and the lid portion 303 thereby. The details will be described later, but when a liquid such as the culture medium for conveyance is held within the space, it is possible to hold the liquid within the second vessel 302 for a long period of time.

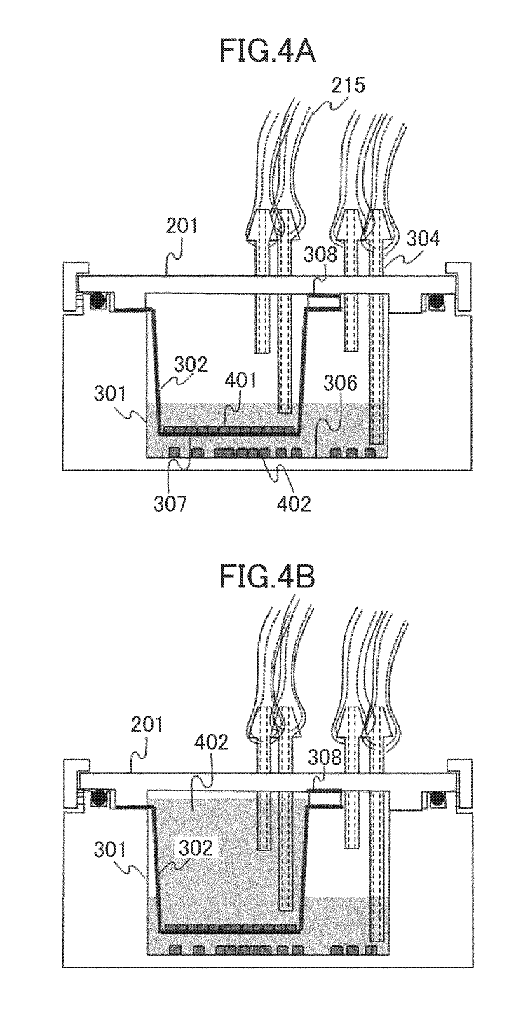

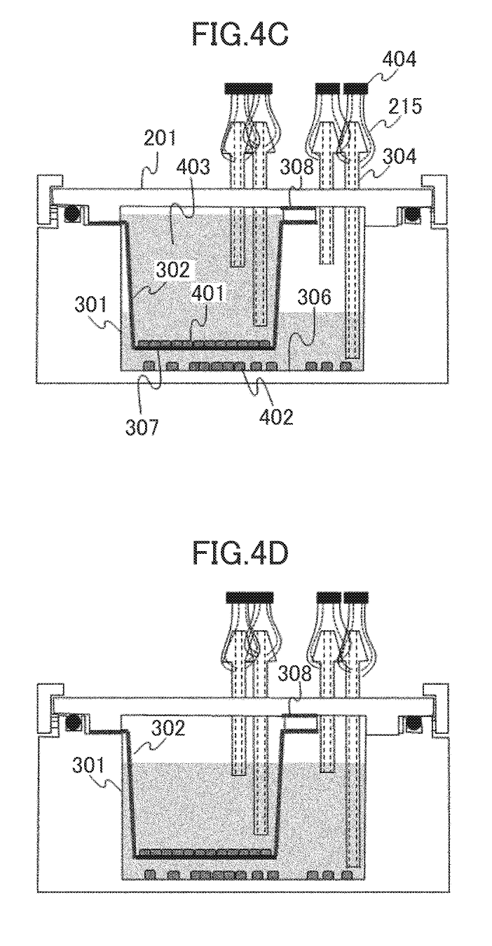

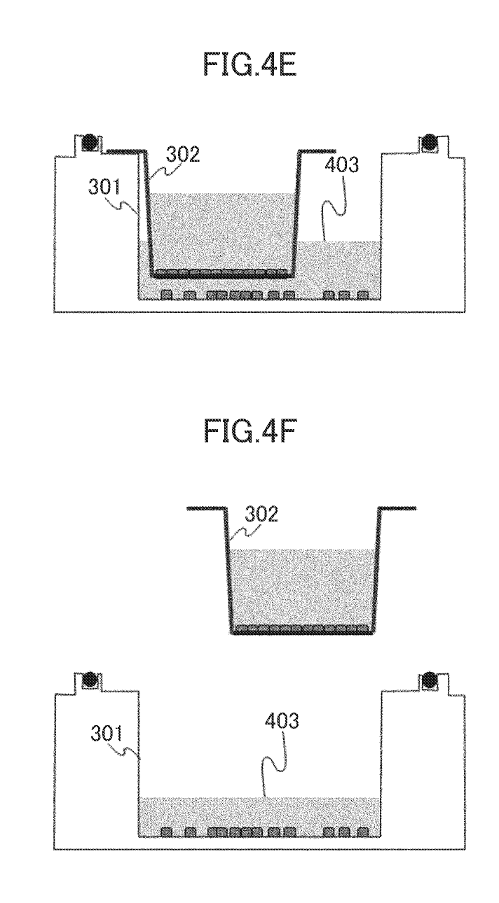

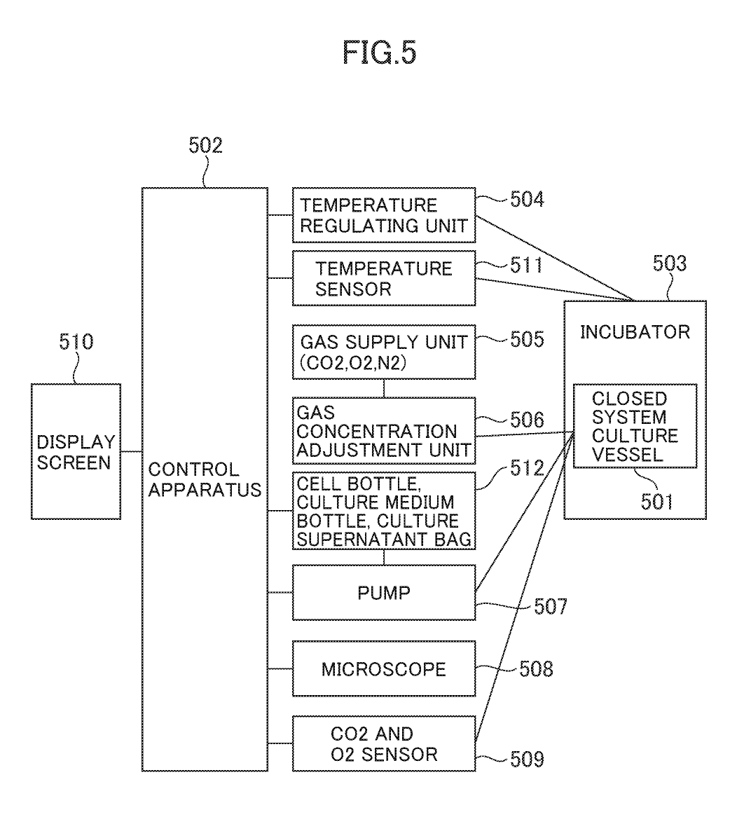

The operation form in each process of the closed system culture vessel 201 of the present embodiment will be explained using FIG. 4A to FIG. 4F. FIG. 4A shows the state when the closed system culture vessel is arranged in the automated culture apparatus and the cells are automatically cultured. Note that 401 shows epithelial cells and 402 shows feeder cells. Cell seeding, culture medium exchange, and gas exchange are performed by the path circuit shown in FIG. 2. Further, the cells are normally observed from the outside of the closed system culture vessel by optical means such as a microscope. Therefore, the closed system culture vessel, specifically, the first vessel 301 has optical properties which do not interfere with the observation, for example, transparency, smoothness, and the like.

FIG. 4B shows a state in which the culture medium for conveyance 403 is filled within the second vessel 302 which is the insert vessel and as preparation after automatic culturing and before conveyance. The culture medium for conveyance 403 is the same culture medium, PBS solution, physiological saline, and the like as that used at the time of culturing. The culture medium is selected in accordance with the type of regenerative tissue, the conveyance time and the like. Further, regarding the amount of the culture medium for conveyance, first, the lower end of the hole 308 described in FIG. 3A and FIG. 3B is the maximum fillable height with respect to the filling amount into the second vessel 302 which is the insert vessel. Even when the culture medium for conveyance is filled in excess thereof, the culture medium for conveyance leaks from the hole 308 into the first vessel 301. The greater the filling amount the inside of insert vessel, the higher the proportion of the liquid phase in the space formed by the insert vessel and the lid portion 303 of the closed system culture vessel 201 becomes. The shear stress generated in the cells during conveyance is determined by the extent of agitation of the culture medium for conveyance. The greater the abovementioned filling amount, the greater the extent of the agitation of the culture medium for conveyance is reduced, and as a result, the shear stress generated in the cells decreases. With respect to the filling amount outside the insert vessel or the amount filled in the first vessel 301, as a rule, it is necessary that there is a gas phase in the first vessel 301. Taking this into consideration, the culture medium is selected in accordance with the type of regenerative tissue, the conveyance time and the like. As more culture medium used for conveyance, it is preferable that there is an abundance of the nutrients for the cells which constitute the regenerative tissues. Further, as described later, if the conveyance time is long, the liquid level moves and the reduction effect of the shear stress decreases, thus, the amount of culture medium is also determined in consideration of the liquid level height after the movement. The breadth of the decrease of the reduction effect of the shear stress becomes smaller as the amount of the culture medium for conveyance within the first vessel 301 is larger.

The point that the amount of the culture medium filled within the first vessel 301 can obtain the advantageous point to a lesser extent is described below. The culture medium for conveyance is filled by the operation of the automated culture apparatus, but as shown in the path circuit of FIG. 2, the filling of the culture medium for conveyance uses a gas which is suitable for cells. As a result, the gas is filled on the inside of the closed system culture vessel 201 during conveyance. This gas is air containing, for example, 5% CO2. Alternatively, gases having higher oxygen concentration may be used only when filling the culture medium for conveyance. The maintenance of the pH of the culture medium for conveyance during conveyance, and the securing of oxygen for use in metabolic activities by cells becomes possible thereby. This advantageous point is excellent in that the gas phase is made to an atmospheric state and the pH fluctuates to the alkaline side, and the oxygen concentration is approximately 20%, compared to the case of opening the closed system culture vessel 201 in a safe cabinet, etc., and filling the culture medium for conveyance without using the automated culture apparatus. Therefore, the lower the amount of culture medium filled within the first vessel 301 is, the more the amount of gas used during filling can increase, and as a result, the advantages in the pH maintenance of the culture medium for conveyance during conveyance and the oxygen supply are obtained. The filling amount into the first vessel is determined taking the above into account.

When conveying the closed system culture vessel of the present embodiment, after the passage tubes 215 coupled to the connector part 304 connected to the second vessel are closed by cutting, after using the connector part 304 connected to the first vessel 310 to feed the gas which is suitable for the cells at a normal pressure or more, and after the passage tubes 215 coupled to the connector part 304 connected to the first vessel are closed by cutting, it is possible to sufficiently maintain the state of the cells over a long period of time by a gas which is suitable for the cells which are sealed in a pressurized state. Namely, after culturing, the culture medium is filled within the second vessel of the closed system culture vessel by the automated culture apparatus, and the inside of the first vessel is brought into a state having a gas phase. The passage tubes are aseptically removed in this state from the passages, and conveyed. At the time of transplantation after conveyance, it is possible to take out the regenerative tissues while avoiding the leakage of the culture medium out of the closed system culture vessel due to the gas phase on the outside of the insert vessel.

FIG. 4C shows the state prior to being conveyed, and FIG. 4D shows the state in which the culture medium for conveyance is moved within the closed system culture vessel 201 after being conveyed over a sufficiently long period of time. In both cases, in FIG. 4A and FIG. 4B, the connector part 304 is arranged in the automated culture apparatus via the passage tube, whereas in FIG. 4C and FIG. 4D, a cut portion 404 is aseptically cut by means such as heat welding of the abovementioned passage tubes 215.

As stated above, the second vessel 302 which is the insert vessel has holes on the porous membrane of the entire culture surface 307b. Further, there are fine holes 308 between the second vessel 302 which is the insert vessel and the lid portion 303. During conveyance, the culture medium for conveyance 403 filled within the insert vessel in FIG. 4C is transferred gradually to the first vessel 301 which is outside the insert vessel by the effect of gravity on the culture medium for conveyance, an inclination provided to the closed system culture vessel, and the movement of the culture medium for conveyance within the closed system culture vessel due to oscillation and impacts. Specifically, in FIG. 4C, the liquid level of the culture medium for conveyance 403 into the second vessel 302 which is the insert vessel is higher than the liquid level into the first vessel 301, but in FIG. 4D, the liquid level inside the insert vessel and inside the first vessel become equal. This is caused specifically by the effect of gravity on the culture medium for conveyance 403, and the state of FIG. 4D indicates that the size of the effect of gravity within the second vessel 302 which is the insert vessel is equal to that within the first vessel 301 due to the liquid levels becoming equal. However, FIG. 4D is a drawing in the case where it is assumed that the closed system culture vessel is sufficiently conveyed over a long period of time.

When the realistic conveyance time is a short time for example, there are cases where the CPC which produces the regenerative tissues and an operating room of a medical institution in which the regenerative medical treatment is performed are in the same site. This is a conveyance time within an hour. Further, when performing conveyance domestically within Japan, if the CPC and the medical institution are in major cities, the conveyance time is within the range of half a day. When the conveyance time is a long period of time, it is possible to assume that the conveyance includes overseas conveyance. Realistically, however, the regenerative tissues are products constituted from cells and which perform metabolic activities, thus, the conveyance time will reduce the activity of the cells to some extent. Therefore, the conveyance time is assumed to be several days at most.

Therefore, as stated above, the case where the regenerative cells are conveyed over a sufficiently long period of time is the state shown in FIG. 4D, but in a realistic conveyance time, the liquid level does not move over that distance. The reason is that the culture medium for conveyance is transferred via the hole of the culture surface 307. In addition, when the regenerative tissues adhere to the culture surface on the inside of the insert vessel, the holes of the culture surface 307 are blocked by the cells, and thus, interfere with the movement of the culture medium for conveyance. Furthermore, even when the liquid level moved from the state of FIG. 4C, the culture medium for conveyance is filled within the second vessel 302 which is the insert vessel during most of the conveyance time, and while the reduction effect of the shear stress gradually decreases, the fact that the shear stress reduces does not change. At the time that the regenerative tissues are taken out, the gas phase is always present within the first vessel 301 even in FIG. 4D, thus, the effect of this point does not change.

FIG. 4E is the state in which the lid portion 303 of the closed system culture vessel 20 is taken out after conveyance for a realistic time. FIG. 4F is the state in which the second vessel 302 which is the insert vessel from into the first vessel 301 of the closed system culture vessel is taken out. Conversely, even if the state after conveyance is as shown in FIG. 4D, the liquid level of the culture medium for conveyance into the first vessel 301 is lower than the upper end of the first vessel 301 as shown in FIG. 4E. Therefore, as shown in FIG. 4F, when the second vessel 302 which is the insert vessel is taken out, even if the culture medium for conveyance spills out from the insert vessel, the spilled portion of the culture medium for conveyance 403 is stored within the first vessel 301, thus, the outer circumference of the closed system culture vessel 201 which is unclean can be prevented from contacting the culture medium for conveyance. As a result, it is possible to take out the second vessel 302 which is the insert vessel while maintaining the cleanliness. Further, when taking out the insert vessel, a part of the culture medium for conveyance into the insert vessel may be transferred by an operation such as inclining the insert vessel. Therefore, the insert vessel which is taken out can be handled in a state in which the culture medium for conveyance which is held on the inside is low, thus, it is possible to prevent the risk that the culture medium for conveyance spills out during handling. In another operation, only the regenerative tissues may be taken out without taking out the insert vessel.

Incidentally, an acceptance inspection may be performed after conveyance. As stated above, the observation of the cells from the outside of the closed system culture vessel 201 by optical means such as a microscope is possible at the time of automatic culturing. Therefore, the cells are observed by the same means after conveyance, and the state after conveyance is verified. It is possible to observe the closed system culture vessel while maintaining the closed property, thus, it is possible to eliminate the risk that the regenerative tissues are biologically contaminated.

FIG. 5 is a block diagram for explaining an example of the functional configuration of the automated culture apparatus including the closed system culture vessel 501 of the present embodiment. Each of the constitutional elements controlled by the control apparatus 502 is an entire configuration diagram connected to the closed system culture vessel 501 arranged on the inside of the incubator 503 corresponding to the incubator 103. Incidentally, it goes without saying that the constitutional element arranged within the incubator 503 is the abovementioned closed system culture vessels 101, 201, or the culture vessel arranged within the automated culture apparatus.

In FIG. 5, the temperature regulating unit 504 for controlling the temperature of the incubator 503, a gas concentration adjustment unit 506 having a gas supply unit 505 for controlling the gas concentration within the closed system culture vessel, a pump 507 arranged within the path circuit for automatically switching the culture medium within the closed system culture vessel 201, a microscope 508 corresponding to the microscope 108 for cell observation for the purpose of controlling the operation of the respective constitutional elements, and a CO2 and O2 sensor 509 are connected to the control apparatus 502.

The control apparatus 502 corresponds to the control unit 102 and the display screen 510 of the control terminal 110 corresponds to the processor and the display screen of the display of an ordinary computer provided with a processor including a Central Processing Unit (CPU), a memory unit, and an input output part including a display and a keyboard, and the like. The control apparatus 502 operates various programs stored in the memory on the CPU as a processor. Therefore, the culturing environment in the incubator 503 can be controlled by the temperature regulating unit 504, the gas supply unit 505, the pump 507, the microscope 508, the CO2 and O2 sensor 509, the gas concentration adjustment unit 506, the cell bottle, culture medium bottle, and culture supernatant bag 512, and the execution of the predetermined culturing processes in the closed system culture vessel 501 is possible.

The gas concentration adjustment unit 506 does not need to be directly connected to the closed-system culture vessel 501. The gas concentration adjustment unit 506 may have a configuration that the temperature regulating unit 504, the gas concentration adjustment unit 506 and the CO2 and O2 sensor 509 are connected to the incubator 503. In such a configuration, it is necessary to supply the gas to the closed-system culture vessel 501 from the outside of the vessel, thus, cell culturing becomes possible by depositing a transparent thin membrane having gas permeability such as PC, PS, and polymethylpentene on a part of the lid portion of the closed-system culture vessel, and performing gas exchange inside the closed-system culture vessel 501.

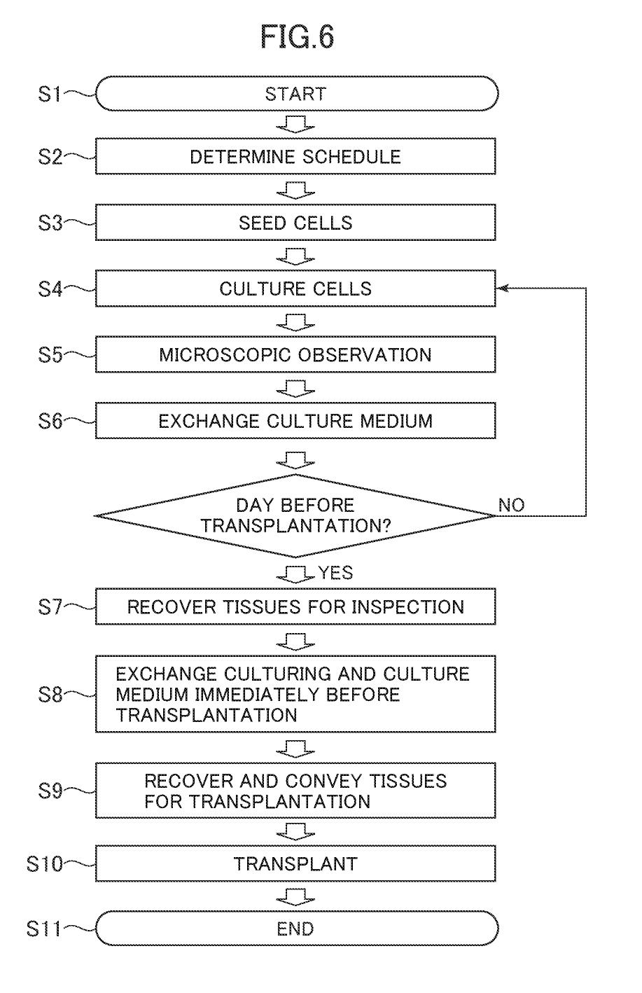

Next, the series of procedures when using the automated culture apparatus including the closed system culture vessel having the aforementioned function, manufacturing the regenerative tissues, and conveying the tissues after being manufactured is shown in FIG. 6.

<Step S1: Start>

The passages including the closed-system culture vessel are arranged in the automated culture apparatus in advance. The passages include the closed system culture vessel, the cell bottle containing the cell suspension, the culture medium bottle containing the culture medium, the culture supernatant bag, etc. for recovering the culture supernatant, and passage tubes which connect the above. After arranging the passages, the normality of the arrangement is verified.

Next, the automated culture apparatus is started. It is started by an operator pressing the start switch on an operation unit in the control apparatus. Incidentally, the inside of the apparatus is a clean environment due to the execution of disinfection or sterilization in advance. It is verified that the internal environment of the automated culture apparatus is appropriate by the operation screen of the display of the control unit. For example, it is verified that the temperature of the incubator is 37° C. These numerical values are not restricted and the temperature can be chosen from a range of 0° C. to 45° C.

<Step S2: Schedule Determination>

The automated culture schedule to perform the automated culture apparatus is determined. The conditions such as the date and volume of the liquid for conducting the operations of cell seeding, culture medium exchange, culture supernatant recovery, gas exchange, microscopic observation, recovery of tissues for inspection, recovery of tissues for transplantation, etc., are inputted from the operation portion of the control unit.

<Step S3: Seeding Cells>

After appropriately opening and closing the solenoid valve, the pump is operated to suck the cell suspension from the cell bottle. The cell suspension is supplied to the closed-system culture vessel. After seeding is terminated in all of the closed-system culture vessels, an actuator mounted on a culture vessel base on which the closed-system culture vessel is arranged is operated, an inclination is provided to the culture vessel base and oscillated, and the cell distribution is made uniform.

<Step S4: Culturing Cells>

Immediately after cell seeding, gas exchange is performed in which a predetermined amount of gas is supplied to the inside of each culture vessel. The gas exchange is executed during the culture period even at a frequency of several times per day. As an example, the gas to be supplied uses air including 5% CO₂ concentration. The flow amount of the gas to each of the closed-system culture vessels from a gas cylinder is controlled by a gas flowmeter, passes through the humidifying bottle, and is supplied in a state saturated with water molecules. The gas which is not needed after being supplied to the closed-system culture vessel is discharged outside the passages via the filter. The filter controls the pressure within the passages in accordance with need. A filter of a quality which, for example, does not allow passage of particle size of 0.22 μm or more is used.

Then, the cells are cultured for a predetermined period of time in a state in which the closed-system culture vessel is left standing horizontally. During the culturing, the temperature is maintained at 37° C. by the incubator. The air inside of the apparatus is constantly agitated by a fan so that the temperature distribution becomes uniform. Incidentally, a particle counter and a viable cell count measurement device can be mounted in the apparatus, and the improvement of the production stability is possible by monitoring the cleanliness.

<Step S5: Microscopic Observation>

A cell image is obtained using a microscope arranged within the automated culture apparatus. A light source arranged in the automated culture apparatus emits light appropriately, and the focus is set on the cells and images are captured. The obtained cell images are stored in a database in the control part, are made available for viewing on the control terminal of the automated culture apparatus, and the state of the cell can be appropriately verified by the operator. Further, other than at the time of automatic cell photographing, the microscope is operated manually by the operator in accordance with need, and the observation and the photographing of the cells are performed.

<Step S6: Culture Medium Exchange>

The culture medium exchange is performed during the culture period at a frequency of once every several days. The culture medium stored in the refrigerator at 4° C. is supplied to the preheated bottle and preheated. First, the old culture medium is discharged from the closed-system culture vessel. At this time, the closed-system culture vessel is inclined by the actuator and the discharge efficiency improves. After the old culture medium is discharged, the new culture medium is supplied promptly into the closed-system culture vessel. The old culture medium is eventually discharged to the culture supernatant bag. The culture supernatant in the culture supernatant bag is recovered according to need, and the growth state of the cells is evaluated by the culture medium component analysis. Incidentally, the culture medium exchange may be performed by means for extruding the new culture medium with the old culture medium kept.

<Step S7: Recovery of Tissues for Inspection>

On the day before transplantation is scheduled, and when a plurality of the closed-system culture vessels are cultured simultaneously, one of the closed-system culture vessels is recovered for inspection. The door of the automated culture apparatus is opened and the passage tubes of the closed-system culture vessel for inspection are aseptically cut and removed by means such as heat welding. The closed-system culture vessels taken out are conveyed to a safe cabinet or outside the CPC, and an inspection is performed promptly. For example, the number of cells of the biological sample, the survival rate, the expression of specific proteins, and the like are evaluated.

<Step S8: Culturing and Culture Medium Exchange Immediately Before Transplantation>

Culturing is performed by the same operation as in Step S4. Moreover, immediately before performing Step S9, the culture medium exchange is performed by the same operation as Step S6. The microscopic observation may also be performed by the same operation as Step S5 according to need.

<Step S9: Recovery and Conveyance of Tissues for Transplantation>

When it is determined as a result of the evaluation in Step S7 that the regenerative tissue are suitable for transplantation, the biological samples are recovered and used for regenerative medical treatment. The culture medium exchange of the culture medium for conveyance is performed by the same operation as in Step S6. At this time, a sufficient amount of the culture medium for conveyance is filled within the insert vessel. The amount of culture medium for conveyance is as shown in FIG. 4B. Next, in the same manner as in S7, the closed system culture vessel is aseptically removed from the passages and taken out of the incubator. Moreover, the closed system culture vessel is stored in a conveyance vessel by a shipping container. The conveyance vessel consists of the heat storage materials, an airtight vessel, packaging and the like, and the specification is selected in accordance with the conveyance route and the time necessary. The influences such as that of the temperature, the pressure, and impacts over the entire distance during conveyance are avoided. After packing the closed system culture vessel in a conveyance vessel, the conveyance vessel is carried outside of the CPC. The conveyance vessel is conveyed by means such as by motor vehicle, trains, airplanes, or by hand to the operating room in accordance with need.

Before the treatment in an operating room, cell observation is performed by a microscope as an acceptance inspection in accordance with need. The closed system culture vessel is transparent which makes microscopic observation possible at the time of automatic culturing, thus, the regenerative tissues are stored as is within the closed system culture vessel, that is, the cleanliness is maintained as is, and microscopic observation of the regenerative tissues is possible as the acceptance inspection. Incidentally, when conveying over a short distance, it is assumed that the state immediately before conveyance will not change much, and thus, the microscopic observation of the regenerative tissues does not need to be performed depending on the decision of the operator.

<Step S10: Transplantation>A

fter arriving at the operating room, the regenerative tissues are taken out from the closed-system culture vessel. When opening the vessel, there is the possibility that the outside of the closed-system culture vessel is adhered with organisms such as bacteria and particles, and thus, the closed-system culture vessel is opened aseptically in order to maintain the cleanliness within the closed-system culture vessel. Initially, the lid portion of the closed system culture vessel is removed. At this time, the inside of the first vessel is in a state having the gas phase. Therefore, if the closed system culture vessel is in a horizontal state and the lid portion is removed, the culture medium for conveyance can be prevented from leaking to the outside of the closed system culture vessel. Further, when the insert vessel is taken out from the first vessel, the leaking of the culture medium for conveyance to the outside of the closed system culture vessel can be similarly prevented by the gas phase within the first vessel which is outside the insert vessel. Alternatively, the culture medium for conveyance filled within the insert vessel is transferred from the insert vessel to the first the culture vessel in advance by inclining the insert vessel when the insert vessel is taken out. It becomes possible thereby to handle outside the closed system culture vessel in a state in which the amount of the culture medium for conveyance into the insert vessel is low. Then, the regenerative tissues are taken out from the insert vessel. As another method, the insert vessel is stored within the closed system culture vessel as is, and the regenerative tissues are directly taken out from the insert vessel.

<Step S11: End>

After the regenerative tissues are taken out, the closed system culture vessel is properly disposed of as medical waste. The automated culture apparatus is brought into a clean state by subjecting to sterilization by a sterilization gas or disinfection by ethanol by taking out the passages used in culturing as well as an appropriate operation to the inside of the apparatus. Various software of the automated culture apparatus is terminated, and the operation of the automated culture apparatus is terminated.

According to the preferred embodiment of the automated culture apparatus including the closed-system culture vessel constituted as stated above, culturing which maintains an aseptic state at the time of culturing is realized, and the avoidance of an influence to cells due to the shear stress during conveyance after being manufactured and the maintenance of the cleanliness of the inside of the closed system culture vessel is possible. As a result, the regenerative medical treatment can be stably performed.

### Second Embodiment

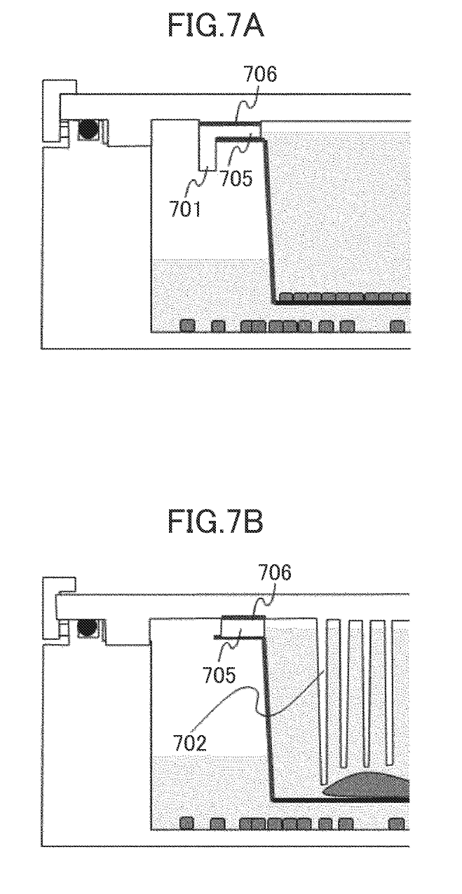

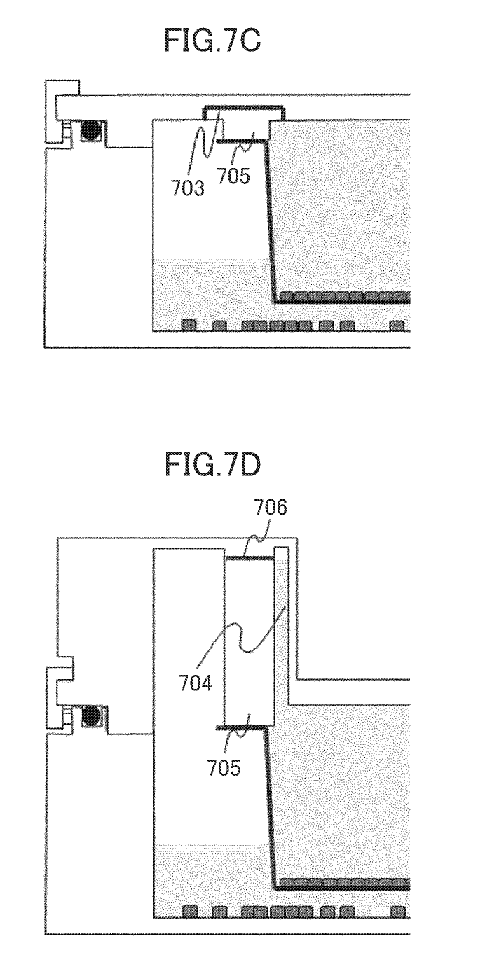

FIG. 7A to FIG. 7D show embodiments of the lid portion of the closed system culture vessel which are different from the first embodiment. In the present embodiment, for the sake of convenience, a convex portion 705 provided with fine holes 706 is shown in a horizontally inverted position relative to FIG. 3A and FIG. 3B, etc., of the first embodiment, and the drawing of the connector part is omitted. FIG. 7A is a drawing in which a splash prevention wall 701 was provided on the convex portion 705 of the lid portion. At the time the regenerative tissues are taken out after conveyance, when removing the lid portion, the splash prevention wall 701 is for the purpose of preventing the splashing of the culture medium for conveyance from the inside of the insert vessel from scattering. It is possible to further improve the maintenance of the cleanliness when the regenerative tissues are taken out after conveyance thereby.

FIG. 7B is a drawing in which a movement control convex portion 702 is provided to the lid portion. The regenerative tissues are not adhered to, or are not firmly adhered to the culture surface after being manufactured by the automated culture apparatus, depending on the type of the regenerative tissue. Cartilage regeneration may be taken up as an example. When conveying such a material, the regenerative tissues move within a space in which the insert vessel and the lid portion form due to the effect of gravity on the regenerative tissues, the inclination provided to the closed system culture vessel, and oscillation and impact, and collide on the inner wall of the insert vessel or the lid portion, and as a result, there is the risk that the regenerative tissues are damaged and the quality declines. In order to prevent this, it is the object to provide the movement control convex portion 702 and control the transfer of the regenerative tissues during conveyance. In the drawing, the movement control convex portion 702 is shown as an example in which a plurality of the convex portions having elongated shapes are arranged, but one convex portion having a thick shape so as to be bundled may be included as another example thereof.

In FIG. 7C, fine through holes 703 are provided in the lid portion instead of the fine holes arranged in the convex portion 705. As described in the first embodiment, the maximum height which may be filled within the second vessel which is the insert vessel is the lower end of the fine holes 308 shown in FIG. 3A and FIG. 3B. Even if the culture medium for conveyance is filled in excess thereof, the culture medium for conveyance will leak from the fine holes 308 into the first vessel 301. Therefore, as shown in FIG. 7C, the fine through holes 703 which pass though the inside of the lid portion may be provided. Therefore, the maximum height fillable within the insert vessel is the lower end of the portion within the insert vessel of the lid portion. In FIG. 7D to be described later, the filling amount of the culture medium for conveyance easily increases by making the height of the closed system culture vessel itself tall, but the embodiment shown in FIG. 7C increases the filling amount of the culture medium for conveyance while having the same height of the closed system culture vessel itself as the first embodiment. Therefore, the height of the closed system culture vessel itself does not change during storage within the conveyance vessel, and thus, the advantageous point that the loading rate is the same can be obtained.

FIG. 7D is a drawing in which a culture medium additional filling unit 704 is provided in the lid portion. Similarly to FIG. 7C, the culture medium additional filling unit 704 has the purpose of increasing the filling amount of the culture medium for conveyance. Furthermore, the liquid level after the filling of the culture medium for conveyance is located above the inside of the space which the second vessel which is the insert vessel and the lid portion forms, and the distance from the culture surface of the insert vessel where the regenerative tissues are arranged is large compared to other embodiments. The agitation of the culture medium for conveyance during conveyance becomes lower, and as a result, the shear stress produced in the regenerative tissues becomes lower. Meanwhile, the height of the closed system culture vessel itself becomes large compared to other embodiments as explained in FIG. 7C, thus, the loading rate to the conveyance vessel decreases.

### Third Embodiment

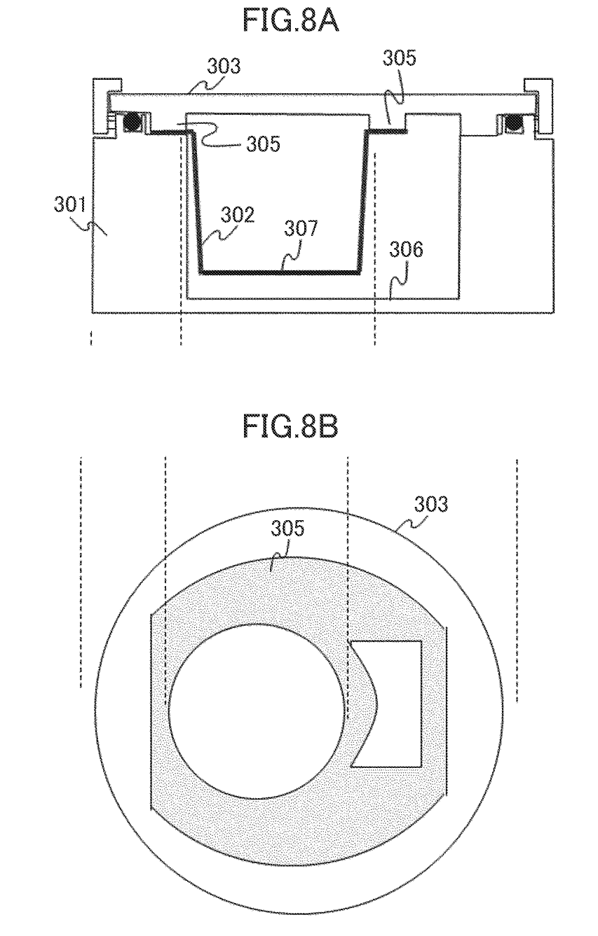

FIG. 8A and FIG. 8B show another embodiment of the closed system culture vessel which is different from the first and second embodiments. A third embodiment is an example of the closed system culture vessel for holding cells provided with a first vessel, a second vessel arranged within the first vessel and having a porous membrane coupling the opening with the first vessel side, and a lid portion for sealing the first vessel, wherein the lid portion forms a closed space with the first vessel, and, the lid portion is in contact with the entire circumference of the opening of the second vessel, and controls the liquid being held within the second vessel from being transferred into the first vessel to make it possible to hold the liquid in the second vessel. Compared to the features shown in FIG. 3A and FIG. 3B, etc., of the first and second embodiments, the third embodiment is different only in that there are no connector part 304 and holes 308, 703, 706, and the other features are the same. The third embodiment has the insert vessel having the regenerative tissues inside after culturing in the culturing by a manual operation using a 6 well plate and the insert vessel which are generally used in culturing by a manual operation.

Namely, the use is assumed when transferring only the second vessel 302 and conveying within the closed system culture vessel by a manual operation. The connector part 304 and the hole 308 are configurations necessary for automatic culturing by the automated culture apparatus, thus, the third embodiment does not have these features.

After culturing by a manual operation which uses a 6 well plate and the insert vessel, the cells are transferred to the inside of a safe cabinet. Initially, the lid portion 303 of the closed system culture vessel is removed within the safe cabinet. Next, the insert vessel within the 6 well plate, i.e., the second vessel 302 is stored within the first vessel 301 of the closed system culture vessel. Moreover, the culture medium for conveyance is filled within the second vessel 302 by a manual operation. Lastly, the closed system culture vessel is made to a closed state by the lid portion 303. The closed system culture vessel is conveyed in this state as in the first embodiment, and the taking out of the regenerative tissues is performed after conveyance.

One feature which is different compared to the first embodiment is that there are no fine holes 308, thus, the speed at which the culture medium for conveyance moves during conveyance from the second vessel 302 through the holes of the culture surface 307 which is a porous membrane decreases. The reason is that there are no fine holes 308 in the configuration of the present embodiment, thus, as the liquid level within the second vessel 302 decreases, the volume of the gas phase produced within the second vessel 302 increases and is in a reduced pressure state, and thus, the effect of gravity operating on the culture medium for conveyance within the second vessel 302 is resisted. As another difference, there is no connector part 304, thus, the closed system culture vessel becomes flatter. As a result, it becomes possible to carry the closed system culture vessel in a more integrated state during conveyance. Further, it becomes easy to increase the contact efficiency with a heat storage material or a cold storage material inside the conveyance vessel, thus, the temperature maintenance within the closed system culture vessel becomes better. Incidentally, in the present embodiment, it is possible to combine with the other examples of the lid portion shown in the second embodiment.

A use method other than when transferring only the insert vessel after the manual operation which used the 6 well plate and the insert vessel which are generally used in culturing by a manual operation will be described below. The second culture vessel 302 which is the insert vessel is kept inserted within the closed system culture vessel in advance. The regenerative tissues are produced by culturing by a manual operation, and only the regenerative tissues are transferred into the closed system culture vessel from which the lid portion had been removed in advance. The subsequent operations are the same as above. In yet another usage method, the biological sample piece which is the raw material of the regenerative tissues, for example, a skin sample, an oral mucosa sample, and the like, or a cornea for corneal transplantation is similarly transferred into the closed system culture vessel in which the second culture vessel 302 which is the insert material was stored in advance. The subsequent operations are the same as above. The usage method described herein is a method for conveying only the regenerative tissues or the biological sample after transferring to the closed system culture vessel.

### Fourth Embodiment

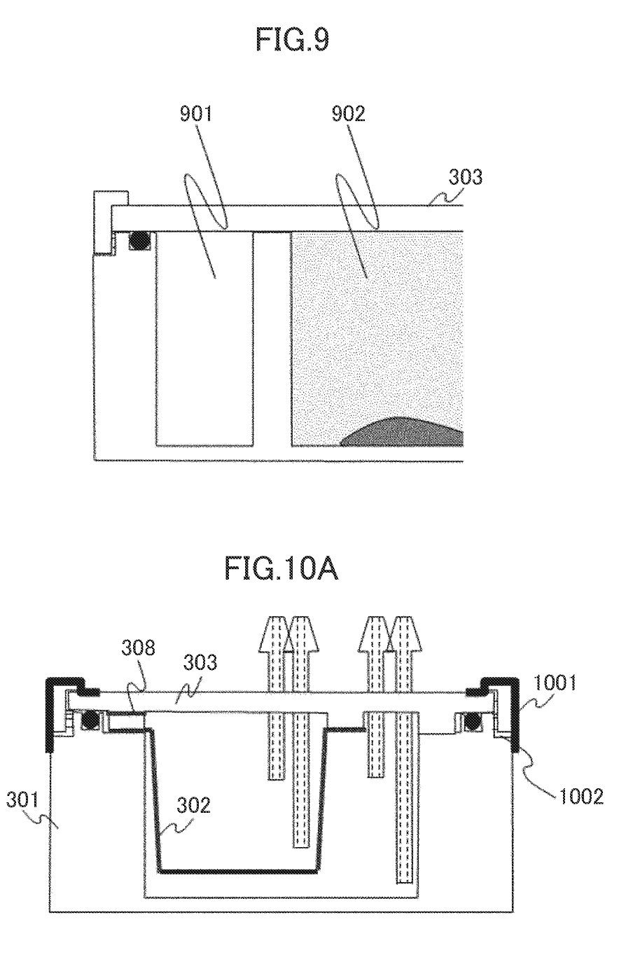

FIG. 9 shows another exemplary configuration of the closed system culture vessel which is different from the first embodiment. The present embodiment is an example of a closed system culture vessel for holding cells of a configuration provided with a first semi-open space, a second semi-open space arranged in order to surround the first semi-open space, and a lid portion for sealing the first semi-open space to form a closed space, wherein the lid portion is in contact with the opening of the second semi-open space over the entire circumference, the transferring of the liquid held within the second semi-open space to the inside of the first semi-open space surrounding the second semi-open space is controlled by the contact with the lid portion and the opening of the second semi-open space to make it possible to hold the liquid in the second semi-open space. A semi-open space means a configuration forming a closed space having an opening in the upper part, and the first and the second semi-open spaces and the lid portion respectively correspond to the first vessel, the second vessel and the lid portion in the aforementioned embodiments.

The use of a region of the closed system culture of the present embodiment is the case where the regenerative tissues are produced by culturing by a manual operation, and only the regenerative tissues are transferred and conveyed within the closed system culture vessel from which the lid portion had been removed in advance, or the case where the biological sample piece which is the raw material of the regenerative tissues, for example, a skin sample, an oral mucosa sample, and the like, or a cornea for corneal transplantation is transferred and conveyed into the closed system culture vessel from which the lid portion had been removed in advance. The method for conveying only the regenerative tissues or the biological sample after transferring to within the closed system culture vessel is the same as the other embodiment.

As shown in FIG. 9, the closed system culture vessel has a first semi-open space 901, a second semi-open space 902, and a lid portion 303. The first vessel 301 and the second vessel 302 which is the insert vessel shown in the first embodiment respectively correspond to the first semi-open space 901 and the second semi-open space, and the lid portion 303 corresponds to the lid portion of the present embodiment. During conveyance, the closed system culture vessel is conveyed in a state in which the culture medium is filled in the second semi-open space 902.

After culturing by a manual operation, the culture vessel, etc., used in the culturing are transferred to the inside of a safe cabinet. Initially, the lid portion of the closed system culture vessel is removed on the inside of the safe cabinet. Next, the regenerative tissues from among the culture vessels are transferred to the second semi-open space 902. Moreover, the culture medium for conveyance is filled into the second semi-open space 902 by a manual operation. Lastly, the closed system culture vessel is brought into a closed state by the lid portion. At this time, even if the culture medium for conveyance spills out and moves into the first semi-open space 901, there are no problems so long as there is a gas phase in the first semi-open space 901 when the lid is mounted. After the lid was mounted, the closed system culture vessel is conveyed in this state similarly to the first embodiment, and the taking out of the regenerative tissues is performed after conveyance.

One feature which is different compared to the first embodiment is that there are no holes connecting the first semi-open space 901 with the second semi-open space 902. Therefore, the culture medium for conveyance filled in the second semi-open space 902 before conveyance continues to be maintained at the same liquid volume after conveyance. In short, compared to the first embodiment, as the conveyance time elapses, the reduction effect of the shear stress does not decrease and remains constant. Further, the closed system culture vessel of the present embodiment also obtains an advantage in that there are fewer parts compared to the first embodiment. In short, the reduction of the production costs is possible due to the production, etc., by injection molding. Incidentally, in the present embodiment, it is also possible to combine other embodiments of the lid portion shown in the second embodiment. Namely, the splash prevention part 701 shown in FIG. 7A may be formed on the lid portion facing the first semi-open space 901, and the movement control convex portion 702 and the culture medium additional filling unit 704 shown in FIG. 7B and FIG. 7D may be formed on the lid portion facing the second semi-open space.

Transferring the liquid held within the second semi-open space 902 into the first semi-open space 901 which is outside of the second semi-open space by the configuration of the present embodiment is controlled by the contact of the lid portion and the opening of the second semi-open space 902, it is possible to hold the liquid in the second semi-open space over a long period of time, the regenerative tissues are stored in the second semi-open space and conveyed in a state in which the culture medium is filled within the second semi-open space, and as a result, it is possible to reduce the shear stress in the regenerative tissues producible by agitating the culture medium for conveyance due to the oscillation and impact generated during conveyance.

### Fifth Embodiment

An operation method of the closed system culture vessel different from in the first embodiment will be explained. In the first embodiment, after culturing by the automated culture apparatus, the gas suitable for the cells is filled in the gas phase within the closed system culture vessel simultaneously with the replacement of the culture medium for conveyance by the operation of the automated culture apparatus, and finally, the passage tubes are aseptically cut by means such as heat welding, and are removed from the automated culture apparatus. In the present embodiment, after filling gas which is suitable for the cells in the gas phase within the closed system culture vessel simultaneously with the replacement of the culture medium for conveyance by the operation of the automated culture apparatus, some of the passage tubes are aseptically cut, the gas suitable for the cells is again filled in the closed system culture vessel, and finally, the remaining passage tubes are aseptically cut by means such as heat welding. The passage tubes to be cut first are tubes other than the passage tubes by which the gas is supplied in the filling of gas suitable for the cells. After these passage tubes are aseptically cut, the gases are supplied by the passage tubes which are not aseptically cut at that point. Compared to the first embodiment, the tubes other than those on the supply side are closed, thus, it is possible to fill the gas in a state pressurized to the atmospheric pressure or more. Then, the remaining passage tubes are aseptically cut. The pH maintenance and the oxygen supply become better compared to the first embodiment thereby. However, pressure is also applied to the regenerative tissues, thus, the present embodiment is effective in the extent that the pressure is not a problem to the quality of the regenerative tissues.

### Sixth Embodiment

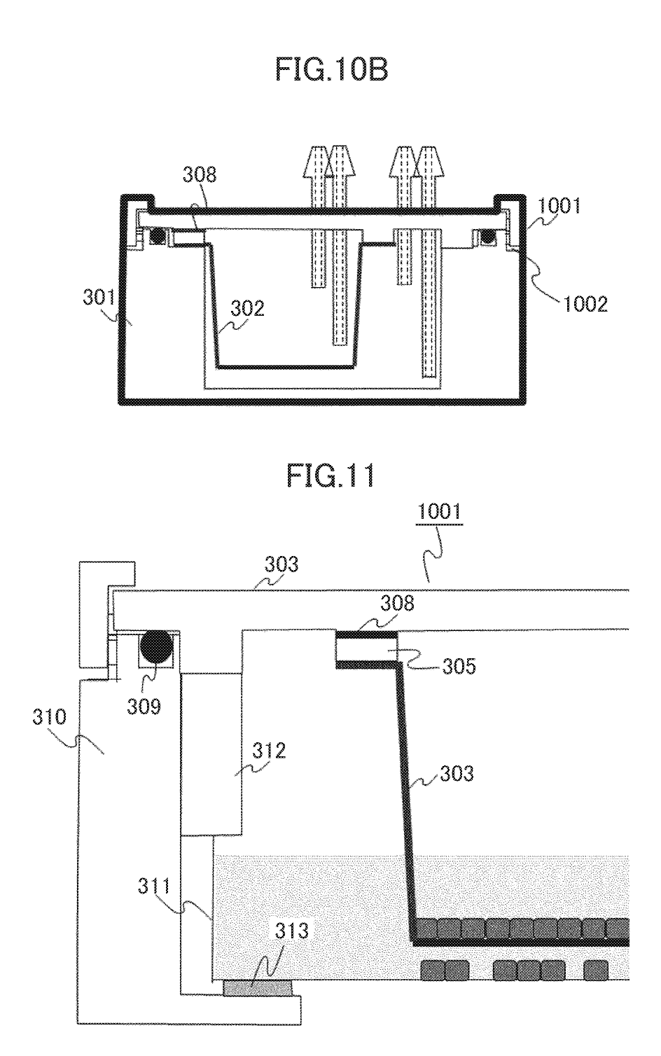

The configuration of the closed system culture vessel shown in FIG. 10A and FIG. 10B is different from the first embodiment. The closed system culture vessel is preassembled and sterilized. A cover member 1001 is arranged in the production process. The cover member 1001 is arranged so as to cover the entire circumference of the boundary of the first vessel 301 and the lid portion 303 of the closed system culture vessel.

As shown in FIG. 3, first, an O-ring and the like is arranged between the first vessel 301 and the lid portion 303 of the closed system culture vessel and sealed in a crimped state by a screw structure, etc. In the present embodiment, however, a space 1002 is further provided between the O-ring and the cover member 1001. The space is provided prior to arranging the closed system culture vessel in the automated culture apparatus, thus, unless the cover member 1001 is removed, the space 1002 maintains the cleanliness. The space 1002 after being taken out remains clean by removing the cover member 1001 in a clean space such as an operating room after conveyance.

During conveyance, the closed system culture vessel is transferred to an unclean space. Therefore, the outer circumference of the closed system culture vessel is clean until the time of production, whereafter it becomes unclean. After conveyance, when taking out the regenerative tissues, the culture medium for conveyance filled within the second vessel is prevented from spilling outside of the first vessel due to the gas phase provided within the first vessel as shown in the first embodiment. In the present embodiment, even if the culture medium for conveyance spilled outside of the first vessel in this process, the space 1002 outside the first vessel remains clean after conveyance, thus, it is possible to prevent the regenerative tissues from being biologically contaminated.

FIG. 10A shows the case where the cover member 1001 arranged between the first vessel 301 and the lid portion 303 of the closed system culture vessel is covered to the side surface of the first vessel 301. FIG. 10B shows the case where the entirety of the closed system culture vessel is covered except for the connector part 304. In the latter case, not only is the space 1002 maintained in a clean state after conveyance, but all of the surfaces of the first vessel 301 are maintained in a clean state after conveyance. Therefore, as stated above, even if the regenerative tissues spill outside of the first vessel in the taking out process after conveyance, the entire outer circumference outside of the first vessel remains clean after conveyance, thus, it becomes easier to prevent the regenerative tissues from being biologically contaminated. However, in this case, the cover member 1001 covers the entire surface of the closed system culture vessel, thus, it is necessary to have optical properties, for example, transparency, smoothness, and the like which do not interfere when observing cells from the outside of the closed system culture vessel by optical means such as a microscope at the time of automatic culturing.

### Seventh Embodiment

FIG. 11 shows the configuration of another embodiment of the closed system culture vessel different from the first embodiment. In the configuration of the closed system culture vessel described for each of the above embodiments, for example, as shown in FIG. 3A and FIG. 3B, the first vessel of the closed system culture vessel was integrally formed with the outside vessel, but by utilizing the culture dish as the first vessel, the outside vessel may be made to another configuration. In FIG. 11, the closed system culture vessel 1101 of the seventh embodiment is different from the first embodiment, and uses a culture dish 311 as the first vessel. Moreover, a culture dish 311 which is the first vessel is held on the outside vessel 310 via the holding member 313, and is fixed by the lid portion 303 via the auxiliary member 312. The other features are the same as the configurations explained in the first embodiment, thus, an explanation has been omitted. In the case of the present embodiment, a commercially available culture dish can be used as the first vessel, and the insert vessel can be used as the second vessel as is. The form of the present embodiment can also be applied to the closed system culture vessels of each of the other embodiments regardless of the first embodiment.

The present invention described above realizes automatic culturing which maintains an aseptic state, and makes it possible to convey the regenerative tissues after the automatic culturing while maintaining the same closed property as at the time of culturing. Thus, the reduction of the shear stress generated due to the agitation of the culture medium for conveyance becomes possible during conveyance, and furthermore, it becomes possible to take out the regenerative tissues from the closed system culture vessel while maintaining the cleanliness during conveyance.

Note that, the present invention is not limited to the aforementioned embodiments, and includes various modification examples. For example, the embodiments have been described in detail to clearly understand the present invention, and are not always limited to ones including all the described configurations. Further, some configurations of a certain embodiment can be replaced with configurations of another embodiment, and configurations of another embodiment can be added to configurations of a certain embodiment. Some of the configurations of each of the embodiments can be added, removed, and replaced with the other configurations.

Furthermore, for the configurations, functions, processing parts, and the like described above, an example is described in which a program that implements some or all of them by a program operation in a CPU is implemented. However, it goes without saying that it may be fine that some or all of them are implemented by hardware by designing some or all of them using an integrated circuit, for example.

The present invention has been described in detail above, but not only the matters described in the claims, but numerous inventions are disclosed in the aforementioned description. A part thereof is listed as follows.

<Listing 1>

A closed system culture vessel which holds cells, comprising:

a first vessel; a second vessel arranged within the first vessel and having a hole coupling with the first vessel side; and a lid portion for sealing the first vessel,

wherein the lid portion forms a closed space with the first vessel, and

the lid portion is in contact with an opening of the second vessel over the entire circumference,

transfer of a liquid held within the second vessel into the first vessel which is outside the second vessel is controlled by the contact between the lid portion and the opening of the second vessel, and

as a result, it is possible to hold the liquid in the second vessel over a long period of time.

<Listing 2>

An automated culture apparatus which uses the closed system culture vessel including the closed system culture vessel which holds cells and a holding member for holding the closed system culture vessel,

wherein the closed system culture vessel has a first vessel, a second vessel arranged within the first vessel and having a hole on the culture surface coupled with the first vessel side, and a lid portion for sealing the first vessel,

the closed system culture vessel and the holding member have a connector part which performs the feeding of the liquid or a gas which is necessary for the culturing,

the automated culture apparatus has the closed system culture vessel and a control unit for controlling the feeding of a liquid or gas via the connector part, and the lid portion forms a closed space with the first vessel,

the lid portion contacts the entire circumference of the opening of the second vessel with the exception of the fine holes coupled with the first vessel side,

transfer of the liquid held within the second vessel into the first vessel which is outside the second vessel is controlled by the contact between the lid portion and the opening of the second vessel,

as a result, it is possible to hold the liquid in the second vessel over a long period of time, and

the gas phase remains in the first vessel, and the liquid is controlled to fill the second vessel.

<Listing 3>

An automated culture apparatus which uses the closed system culture vessel including the closed system culture vessel which holds cells and a holding member for holding the closed system culture vessel,

wherein the closed system culture vessel has a first vessel, a second vessel arranged within the first vessel and having a hole on the culture surface coupled with the first vessel side, and the lid portion for sealing the first vessel,

the closed system culture vessel and the holding member have a connector part which performs the feeding of the liquid or a gas which is necessary for the culturing,

the automated culture apparatus has the closed system culture vessel and a control unit for controlling the feeding of a liquid or a gas via the connector part, and the lid portion forms a closed space with the first vessel,

the lid portion contacts the entire circumference of the opening of the second vessel with the exception of the fine holes coupled with the first vessel side,

the control unit transfers the gas within the first vessel by the fine holes coupled with the first vessel side arranged in the second vessel and simultaneously supplies or discharges the cell suspension or culture medium by the connector part connected to the first vessel so as to equalize the pressure on the inside of the second vessel, the gas in the second vessel is transferred out of, or transferred into the closed system culture vessel by the connector part connected to the second vessel, and as a result, the closed system culture vessel is maintained at atmospheric pressure.

<Listing 4>

An automated culture apparatus which uses the closed system culture vessel including the closed system culture vessel which holds cells and the holding member for holding the closed system culture vessel,

wherein the closed system culture vessel has a first vessel, a second vessel arranged within the first vessel and having a hole coupled with the first vessel side, and a lid portion for sealing the first vessel,

the closed system culture vessel and the holding member have a connector part which performs the feeding of the liquid or a gas which is necessary for the culturing,

the automated culture apparatus has the closed system culture vessel and a control unit for controlling the feeding of a liquid or gas via the connector part, and the lid portion forms a closed space with the first vessel,

the lid portion contacts the entire circumference of the opening of the second vessel with the exception of the fine holes coupled with the first vessel side, and

the control unit transfers the gas within the first vessel by the fine holes connected with the first vessel side arranged in the second vessel and simultaneously supplies or discharges the cell suspension or culture medium by the connector part connected to the second vessel so as to equalize the pressure on the inside of the second vessel, the gas in the first vessel is transferred out of, or transferred into the closed system culture vessel by the connector part connected to the first vessel, and as a result, the closed system culture vessel is maintained at atmospheric pressure.

<Listing 5>

An automated culture apparatus which uses the closed system culture vessel including the closed system culture vessel which holds cells and the holding member for holding the closed system culture vessel,

wherein the closed system culture vessel has a first vessel, a second vessel arranged within the first vessel and having a hole coupled with the first vessel side, and a lid portion for sealing the first vessel,

the closed system culture vessel and the holding member have a connector part which performs the feeding of the liquid or a gas which is necessary for the culturing,

the automated culture apparatus has the closed system culture vessel and a control unit for controlling the feeding of a liquid or a gas via the connector part, and the lid portion forms a closed space with the first vessel,

the lid portion contacts the entire circumference of the opening of the second vessel with the exception of the fine holes coupled with the first vessel side,

the control unit transfers the gas within the first vessel by the fine holes coupled with the first vessel side arranged in the second vessel and simultaneously supplies the culture medium for conveyance to the height of the fine holes coupled with the first vessel side arranged within the second vessel by the connector connected with the second vessel so as to equalize the pressure on the inside of the second vessel,

the gas in the first vessel is transferred outside of the closed system culture vessel by the connector part connected to the first vessel, and

as a result, using the automated culture apparatus used in culturing makes it possible to fill the culture medium for conveyance while maintaining the cleanliness on the inside without opening the closed system culture vessel.

<Listing 6>

A closed system culture vessel which holds cells, comprising:

a first vessel; a second vessel arranged within the first vessel and having a hole coupling with the first vessel side; and a lid portion for sealing the first vessel,

wherein the lid portion forms a closed space with the first vessel,

the lid portion is in contact with the opening of the second vessel over the entire circumference, and has a convex portion in which a lower end is below an upper end of the opening of the second vessel,

transfer of a liquid held within the second vessel into the first vessel which is outside the second vessel is controlled by the contact between the lid portion and the opening of the second vessel,

as a result, it is possible to hold the liquid in the first vessel for a long period of time, and

due to the convex portion of the lid portion, the culture medium for conveyance is transferred from the second vessel when the regenerative tissues are taken out after conveyance into only the first vessel without leaking outside the passages.

<Listing 7>

A closed system culture vessel which holds cells, comprising:

a first vessel; a second vessel arranged within the first vessel and having the hole coupling with the first vessel side; and a lid portion for sealing the first vessel,

wherein the lid portion forms a closed space with the first vessel,

the lid portion is in contact with an opening of the second vessel over the entire circumference, and has a convex portion on the peripheral side inner than the portion which contacts the opening of the second vessel over the entire circumference, and the convex portion has a length which reaches to at most the upper end of the regenerative tissues during conveyance of the regenerative tissues,

transfer of the liquid held within the second vessel into the first vessel which is outside the second vessel is controlled by the contact between the lid portion and the opening of the second vessel,

as a result, it is possible to hold the liquid in the second vessel over a long period of time, and

the convex portion of the lid portion prevents the regenerative tissues from moving during conveyance due to oscillation, impacts, and the inclination of the conveyance vessel during conveyance, and prevents collision and damage to the inner wall within the second vessel.

<Listing 8>

A closed system culture vessel which holds cells, comprising:

a first vessel; a second vessel arranged within the first vessel and having a hole coupling with the first vessel side; and a lid portion for sealing the first vessel,

wherein the lid portion forms a closed space with the first vessel,

the lid portion is in contact with the opening of the second vessel over the entire circumference, and the lid portion has fine holes through which the gas penetrating to the inside of the lid portion can pass,

transfer of the liquid held within the second vessel into the first vessel which is outside the second vessel is controlled by the contact between the lid portion and the opening of the first vessel,

as a result, it is possible to hold the liquid in the second vessel over a long period of time, and

it is possible to increase the amount of culture medium for conveyance that can be filled into the second vessel by arranging the position of the fine holes of the lid portion upward in the vertical direction.

<Listing 9>

A closed system culture vessel which holds cells, comprising:

a first vessel; a second vessel arranged within the first vessel and having a hole coupling with the first vessel side; and a lid portion for sealing the first vessel,

wherein the lid portion forms a closed space with the first vessel,

the lid portion is in contact with an opening of the second vessel over the entire circumference, and the vicinity of the portion in contact with the second vessel on the side of the lid portion in the space which the lid portion forms with the second vessel is longer upwards in the vertical direction than the portion close to the center, and further has fine holes through which the gas may pass above this region, and

transfer of a liquid held within the second vessel into the first vessel which is outside the second vessel is controlled by the contact between the lid portion and the opening of the second vessel,

as a result, it is possible to hold the liquid in the second vessel over a long period of time, and

it is possible to increase the reduction effect of the shear stress by filling the culture medium for conveyance to the region of the lid portion compared to when there is no culture medium for conveyance.

<Listing 10>

An automated culture apparatus which uses the closed system culture vessel including the closed system culture vessel which holds cells and the holding member for holding the closed system culture vessel,

wherein the closed system culture vessel has the first vessel, a second vessel arranged within the first vessel and having a hole coupled with the first vessel side, and a lid portion for sealing the first vessel,

the closed system culture vessel and the holding member have a connector part which performs the feeding of the liquid or the gas which is necessary for the culturing,

the automated culture apparatus has the closed system culture vessel and a control unit for controlling the feeding of a liquid or a gas via the connector part, and the lid portion forms a closed space with the first vessel,

the lid portion contacts the entire circumference of the opening of the second vessel with the exception of the fine holes coupled with the first vessel side,

the passage tubes coupled to the connector part may be closed by means such as heat welding to make it impossible to perform the feeding of the liquid or the gas,

the control unit transfers the gas within the first vessel by the fine holes coupled with the first vessel side arranged in the second vessel and simultaneously supplies the culture medium for conveyance to the height of the fine holes coupled with the first vessel side arranged within the second vessel by the connector connected with the second vessel so as to equalize the pressure on the inside of the second vessel,

the gas in the first vessel is transferred outside of the closed system culture vessel by the connector part connected to the first vessel,

after the passage tubes coupled to the connector part connected to the second vessel are closed, the gas suitable for the cells is fed at a normal pressure or more by the connector part connected to the first vessel,

as a result, using the automated culture apparatus used in culturing makes it possible to fill the culture medium for conveyance while maintaining the cleanliness on the inside without opening the closed system culture vessel, and

the state of the cells is satisfactorily maintained over a long period of time due to the gas suitable for the cells sealed in a pressurized state.

<Listing 11>

A closed system culture vessel which holds cells, comprising:

a first vessel; a second vessel arranged within the first vessel and having the hole coupling with the first vessel side; and a lid portion for sealing the first vessel,

wherein the lid portion forms a closed space with the first vessel,

the lid portion is in contact with an opening of the second vessel over the entire circumference,

the transfer of a liquid held within the second vessel into the first vessel which is outside the second vessel is controlled by the contact between the lid portion and the opening of the second vessel, and it is possible to hold the liquid in the second vessel over a long period of time,

has a cover member forming a space outside the portion coupling the first vessel with the lid portion, and the inside of the space is also in a sterilized state by being arranged before being manufactured and sterilized in advance, and

the cover member maintains the sterilized state even after conveyance by being removed when the regenerative tissues are taken out after conveyance, and as a result, the cover member makes it possible to maintain the cleanliness of the regenerative tissues as long as the culture medium for conveyance leaks only to the space for which the cleanliness is maintained after conveyance even if the culture medium for conveyance leaks from the second vessel and the like to the outside when the regenerative tissues are taken out.

### LIST OF REFERENCE SIGNS

100 culture vessel unit
101, 201, 501, 1101 closed system culture vessel
102 control unit
103, 503 incubator
105, 209, 505 gas supply unit
108, 508 microscope
110 control terminal
112, 205 cell bottle
113, 207 culture medium bottle
114, 211 culture supernatant bag
115, 206 refrigerator
116 passage part
117 liquid feeding mechanism
202 solenoid valve
203 pump
204 filter
208 preheating mechanism
210 humidifying bottle
212 stage
213 microscope observation unit
214 light irradiation portion
215 passage tubes
301 first vessel
302 second vessel
303 lid portion
304 connector part
305, 705 the convex portion
306, 307 culture surface
308, 706 fine holes
309 packing unit
310 outside vessel
311 culture dish
312 auxiliary member
313 support member
401 epithelial cells
402 feeder cells
403 culture medium for conveyance
404 cut portion
502 control apparatus
504 temperature regulating unit
506 gas concentration adjustment unit
507 pump
509 CO2 and O2 sensor
510 display screen
511 temperature sensor
512 cell bottle, culture medium bottle, culture supernatant bag
701 splash prevention wall
702 movement control convex portion
703 fine through hole
704 culture medium additional filling unit
901 first semi-open space
902 second semi-open space
1001 cover member
1002 space.

1. A closed system culture vessel for holding cells, comprising:
a first vessel; a second vessel arranged within the first vessel and having a porous membrane for connecting an opening with the first vessel side; and a lid portion for sealing the first vessel,
wherein the lid portion forms a closed space with the first vessel, and the lid portion is in contact with the opening of the second vessel over the entire circumference, thus, controlling transfer of a liquid held within the second vessel into the first vessel to make it possible to hold the liquid in the second vessel.

2. The closed system culture vessel according to claim 1,
wherein the lid portion has:
a connector part for feeding the liquid or a gas to the first vessel and the second vessel; and
a convex portion contacting the opening of the second vessel.

3. The closed system culture vessel according to claim 2,
wherein the convex portion is provided with fine holes between the first vessel and the second vessel.

4. The closed system culture vessel according to claim 3,
wherein the first vessel has a culture surface on the bottom surface thereof and the convex portion is provided with a splash prevention wall extending along the culture surface of the first vessel.

5. The closed system culture vessel according to claim 3,
wherein the lid portion is provided with a movement control convex portion extending along the porous membrane of the second vessel.

6. The closed system culture vessel according to claim 3,
wherein the lid portion is provided with a culture medium additional filling unit.

7. The closed system culture vessel according to claim 3, comprising a cover member forming a space outside a portion coupling the first vessel with the lid portion.

8. The closed system culture vessel for holding cells comprising:
a first semi-open space; a second semi-open space arranged in order to surround the first semi-open space; and a lid portion for sealing the first semi-open space,
wherein the lid portion forms a closed space with the first semi-open space,
the lid portion is in contact with the opening of the second semi-open space over the entire circumference,
transferring of the liquid held within the second semi-open space into the first semi-open space surrounding the second semi-open space is controlled by the contact between the lid portion and the opening of the second semi-open space, and makes it possible to hold the liquid in the second semi-open space.

9. The closed system culture vessel according to claim 8,
wherein a splash prevention part is formed in the lid portion facing the first semi-open space, or a movement control convex portion or a culture medium additional filling unit is formed in the lid portion facing the second semi-open space.

10. A conveyance method of the closed system culture vessel according to claim 3,
wherein a culture medium for conveyance is supplied into the second vessel to the height of the fine holes, and
conveyance is performed after closing the passage tubes coupled to the connector part.

11. The conveyance method of the closed system culture vessel according to claim 10,
wherein the culture medium for conveyance is supplied to the second vessel to the height of the fine holes, and
after the passage tubes coupled to the connector part connected to the second vessel are closed, the gas which is suitable for the cells are fed at a normal pressure or more from the connector part connected to the first vessel, and conveyed after the passage tubes coupled to the connector part connecting to the second vessel are closed.

12. An automated culture apparatus for culturing cells, comprising:
a holding part for holding a liquid or a gas required for culturing;
a closed system culture vessel for holding the liquid or the gas supplied from the holding part;
a passage part provided with a plurality of passages for connecting the holding part with the closed system culture vessel;
a control unit for controlling the passage part, and for controlling so as to supply or discharge the liquid or the gas from the holding part to the closed system culture vessel,
wherein the closed system culture vessel is provided with a first vessel, a second vessel arranged within the first vessel and having a porous membrane coupling the opening with the first vessel side, and a lid portion for sealing the first vessel,
the lid portion has a connector part for performing the supply or discharge of the liquid or the gas to the first vessel and the second vessel, and
the lid portion forms a closed space with the first vessel, and the lid portion is in contact with the opening of the second vessel over the entire circumference, controlling transfer of the liquid supplied and held within the second vessel to the first vessel to make it possible to hold the liquid in the second vessel.

13. The automated culture apparatus according to claim 12,
wherein the lid portion has a convex portion which is in contact with the opening of the second vessel, and the second vessel is in contact with the opening in the convex portion over the entire circumference.

14. The automated culture apparatus according to claim 13,
wherein the convex portion has fine holes between the first vessel and the second vessel, and
the gas supplied into the first vessel by the fine holes is transferred so as to equalize the pressure on the inside of the second vessel.

15. The automated culture apparatus according to claim 14, comprising a cover member forming a space on the outside of the portion coupling the first vessel with the lid portion.
